(19) United States
(12) Patent Application Publication
(10) Pub. No.: US 2002/0188216 A1
(43) Pub. Date: Dec. 12, 2002

(54) Head mounted medical device

(76) Inventors: Hani Akram Kayyali, Shaker Heights, OH (US); Raymond Lloyd Yoder, Willoughby, OH (US)

Correspondence Address: PEARNE & GORDON LLP, 526 SUPERIOR AVENUE EAST, SUITE 1200, CLEVELAND, OH, 44114-1484, US

(21) Appl. No.: 10/138,165

(22) Filed: May 3, 2002

Related U.S. Application Data
(60) Provisional application No. 60/288,377, filed on May 3, 2001.

(51) Int. Cl. A61B 5/04

(52) U.S. Cl. 600/544

(57) ABSTRACT

A head mounted medical device for obtaining and processing EEG, EKG, and EOG/EMG signals from a wearer. The device utilizes electrodes organized into multiple electrode assemblies for ease of use and replacement, with the electrode assemblies being removably connected to the headband. The device includes processing and conditioning circuitry in the headband, the processing and conditioning including amplification, filtering, A/D conversion, and multiplexing of the analog signals generated by the electrodes, reducing noise influences and improving the handling and mobility of the device. The device connects to an external receiving device that then monitors and/or records the resulting serial, digital, and multiplexed data signal. The external device might also provide power and/or commands to the medical device.

### BACKGROUND OF THE INVENTION

This application claims the benefit of provisional application serial No. 60/288,377, filed on May 3, 2001, and incorporated herein by reference.

The invention generally relates to a wearable medical signal detection device and, more particularly, to a head mounted medical device capable of detecting, collecting, and/or processing biosignals and biometric data which are provided by various biometric sensors, devices, and transducers, all of which can be worn by a patient outside the confines of medical centers and hospitals.

Wearable ambulatory medical devices are becoming more and more important to the medical care of human beings. As computer aided analysis and automated collection of medical data becomes more and more prevalent, it is becoming desirable to collect biometric medical data over long periods of time, frequently while the patient is away from a hospital or medical center, and often while the patient is going about his or her normal daily routines. It is also important to increase the fidelity of the signal being detected as it impacts diagnosis directly. It is also important to minimize any obstruction or interference with the patient user's motion or activities.

Wearable medical devices are useful in the diagnosis and treatment of certain heart conditions, epilepsy and other brain disorders, sleeping disorders, and other medical disorders requiring biometric monitoring and/or data collection. Other medical applications include military (such as monitoring soldier performance in the field or in action), or sports (such as monitoring muscle contraction or activity). Consequently, it would be useful to collect high fidelity, unobtrusive biometric information by observing and recording biosignals and other biometric data relating to the various medical conditions being evaluated.

EEGs, ECGs/EKGs, EOG/EMG (eye movements), and other biosignal data can be useful in monitoring the patient's condition over long periods of time, without interfering with the patient's daily routines. There is a need in the art for a means to record biosignal and biometric data for long periods of time in a manner that does not disrupt the patient's routine, but at the same time provides high signal quality and/or presents data in a useful and practical way to the relevant medical care specialists. Recording devices for biomedical data are being developed. A co-pending application for a Biosignals Recorder, application serial No. 10/116,872, filed on Apr. 5, 2002 by Fernando Casas et al., inventors, incorporated herein by reference, is one example of such a device. Thus, there is a need for medical transducer units to collect the medical data utilized by such ambulatory recording devices such as the Biosignals Recorder.

Existing signal detection medical devices are not ideal because they often use conventional rigid printed circuit board technology making the devices bulky and inflexible. Current devices also use traditional transducer interfaces which mainly consist of multiple, long lead wires (the leads connect the transducer with the signal detection devices). Since these leads are long and can be numerous (more than 32 electrodes can be used for diagnosing epilepsy), patient mobility is limited making the use of such signal detection devices undesirable.

Patient mobility is important because extended time monitoring is often needed for capturing infrequent, but important, physiological events. Also, the long and numerous lead wires degrade signal quality because they serve as antennas making them susceptible to electric and magnetic interference. In some applications there is a need to place the signal detection and acquisition circuitry on the head such as for diagnosing epilepsy or sleep disorders. In that case, a flexible and light signal detection and acquisition circuitry is needed. This application will discuss the head mounted device although other embodiments are possible with the same technology. However, mobility itself can cause problems, such as providing DC shifts in the signals due to cable or wire movement caused by patient movement.

Furthermore, because electrodes are often embedded with the electronics, many current devices tend to be large and uncomfortable to wear. Also, the electrodes required for these headsets tend to be of different material than the widely used conventional electrodes, leading to unforeseen results and/or skin abrasion that are intolerable to the patient. Also, these devices often use total flexible printed circuit boards for their circuitry. Total flexible boards compromise the reliability of component placements and signal continuity due to the potential of component dislodgment away from the flexible substrate. This is especially true for boards that are exposed to continuous bending such as headsets. Also, they often do not include two important features often useful to any EEG procedure: electrode check and signal-test capability.

It is desirable to develop a new head mounted signal detection and acquisition device that can maintain ease of electrode connections, while offering the needed electrode protection (housing) and minimized tethering.

Another variation of the invention is to develop a wearable device, not as a head mounted instrument, but around the waist like a belt. Although the electrode housing protection advantage would not be satisfied in that form factor, minimal tethering would. As a belt worn device, ambulatory patients will no longer have to carry a jack box, which is a device often carried by patients in addition to the recorder. The wearable belt will serve as combined jack box and data acquisition system that directly attaches to the recorder and the entire system can be worn as a belt freeing patients hands and shoulders. This would help patients restore their daily routine activities.

### SUMMARY OF THE INVENTION

1) Provided is a head mounted medical device comprising an electrode for outputting an electrode signal; a headband circuit for inputting and conditioning the electrode signal into a headband data output signal for output by the medical device into an external receiving device.

The medical device has at least one module including some portion of the headband circuit, and a flexible substrate for mounting the at least one module thereon, wherein the flexible substrate can be formed into a loop for mounting on the head of a user.

2) Also provided is a head mounted medical device comprising a plurality of electrodes, each electrode for outputting an electrode signal; and a headband circuit for inputting the electrode signals, wherein the headband circuit amplifies, filters, and multiplexes the electrode signals, further generating a headband data output signal therefrom for output by the device into an external receiving device.

The medical device also comprising an electrode assembly including two or more electrodes; a plurality of modules, each module including a portion of the headband circuit; wherein the electrode assembly can be mechanically and electrically removably connected to at least one of the plurality of modules; and a flexible substrate for mounting the plurality of modules thereon. The flexible substrate electrically connects at least some of the plurality of modules together, wherein the flexible substrate can flex, allowing the flexible substrate to be formed into a loop for mounting on the head of a user.

3) Further provided is a head mounted medical device comprising a controller/processor and a plurality of channels, each channel including an electrode for outputting an electrode signal; and a conditioning circuit for inputting and conditioning the electrode signal. The conditioning circuit is also for outputting a conditioned electrode signal into an input of the controller/processor.

The controller/processor processes the conditioned electrode signal into a processed electrode signal and the controller/processor multiplexes each processed electrode signal together into a headband data output signal for output by the device into an external receiving device.

The medical device further comprising at least one electrode assembly including two or more electrodes and an electrode connector, and a plurality of modules, each module including the controller/processor and/or one or more of the conditioning circuits.

The electrode connector can be mechanically and electrically removably connected to a corresponding headband connector mounted on at least one of the plurality of modules. A flexible substrate is included in the device for mounting the plurality of modules thereon, the flexible substrate electrically connecting at least some of the plurality of modules together, wherein each of said plurality of modules is mounted some distance from another of said modules, creating gaps between the modules. The gaps allow the flexible substrate to flex at the gaps, allowing the flexible substrate to be formed into a loop for mounting on the head of a user.

4) Still further provided is a head mounted medical device comprising a reference electrode for mounting on the scalp, head, or body of a user and also for outputting a reference signal; a ground electrode for mounting on the scalp, head, or body of the user, the ground electrode connected to a ground plane of the medical device; a controller/processor; a power conditioning circuit for conditioning and distributing power to the medical device; and a plurality of channels.

Each channel includes an EEG electrode for mounting on the scalp of the user and also for outputting an analog electrode signal; an impedance check circuit for outputting an impedance test signal to the EEG electrode to determine an EEG electrode impedance of the EEG electrode, wherein the controller/processor monitors the EEG electrode impedance; a conditioning circuit for inputting and conditioning the analog electrode signal, said conditioning including amplifying, filtering, and clamping a DC offset, with the reference signal also being input to the conditioning circuit. The conditioning circuit is also for outputting a conditioned electrode signal into an input of the controller/processor, wherein the controller/processor converts the conditioned electrode signal into a digital electrode signal. Each channel also includes a signal test circuit for outputting a test signal into the conditioning circuit to determine an amplification gain of the conditioning circuit.

The controller/processor multiplexes each digital electrode signal together into a headband data output signal for output by the medical device into an external receiving device, and the controller/processor is also capable of receiving commands from the external receiving device.

The medical device also comprising a plurality of electrode assemblies, each electrode assembly including two or more EEG electrodes and an electrode connector. The medical device also comprising a plurality of modules, each module including at least one printed circuit board; each module also including, mounted on the at least one printed circuit board, one or more of the group consisting of the impedance check circuits, the signal test circuits, the conditioning circuits, the power conditioning circuit, and the controller/processor.

The electrode connector can be mechanically and electrically removably connected to a corresponding headband connector mounted on at least one of the plurality of modules.

A flexible substrate is included in the medical device for mounting the plurality of modules thereon, the flexible substrate having a first end and a second end, the flexible substrate electrically connecting at least some of the plurality of modules together, wherein each of said plurality of modules is mounted some distance from another of said modules, creating gaps between the modules, the gaps allowing the flexible substrate to flex at the gaps, allowing the flexible substrate to be formed into a loop for mounting on the head of the user, wherein the first end and the second end are removably connected together using an adjustable fastening means for securing the first end to the second end, the adjustable fastening means allowing the flexible substrate to be adjusted to fit the head of the user. Also included in the medical device is a flexible headband cover for covering and protecting the modules and the flexible substrate.

### BRIEF DESCRIPTION OF THE DRAWINGS

FIG. 1 is an illustration of the headband of the Head Mounted Medical Device;

FIG. 2 is an illustration of the headband without its external headband covering, thus showing an interior view of the headband;

FIG. 3 is an illustration of the Head Mounted Medical Device as it might be worn by a user;

FIG. 4 is an illustration of one embodiment of an electrode assembly used by the device;

FIG. 5 is a block diagram representing the major electrical components of the Head Mounted Medical Device;

FIG. 6 is a block diagram representing some of the major electrical components of the Head Mounted Medical Device in greater detail; and

FIG. 7 is a circuit diagram showing an embodiment of the conditioning circuit.

### DETAILED DESCRIPTION OF THE PREFERRED EMBODIMENTS OF THE INVENTION

The Head Mounted Medical Device is a versatile signal detection and data acquisition device that measures physiological signals. Such physiological signals include electroencephalogram (EEG) signals and even electrocardiogram (EKG/ECG) and eye movement (EOG/EMB) signals, among others, that are produced on the surface of the body (chest area for EKG/ECG and head/scalp for EEG). Long term recording of these physiological signals is often needed to detect signal abnormalities that are linked to disorders such as cardiac arrhythmia (EKG/ECG) or epilepsy (EEG), among others, that may otherwise be difficult to detect over a short period of time.

The Head Mounted Medical Device output can alternatively or additionally connect to an external receiving device, such as a data recorder, a PC, or a monitoring device. Alternatively, the recording unit might connect to a PC or monitor device. An example of a recording device is contained in the co-pending Biosignals Recorder referenced in the Background section (and incorporated herein by reference).

The Head Mounted Medical Device preferably outputs the electrode data in digital form. However, it might also carry commands from the external device back to the Head Mounted Medical Device. These commands could be used to ensure proper data transmission (e.g., checksum) or to perform electrode continuity checks or signal test procedures to ensure proper signal gains.

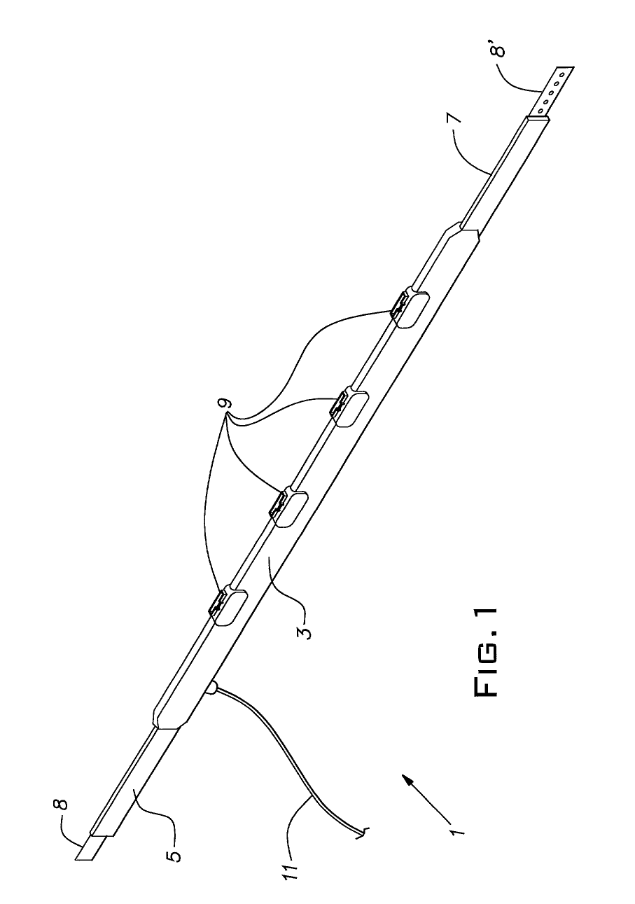

FIG. 1 shows an embodiment of a portion of the device, a headband 1, as it might appear before it would be fitted to a patient's scalp, for example. The headband 1 contains electrical components and structures encased in a headband covering 3 to protect the headband components and provide a comfortable fit. The headband covering 3 could be composed of elastomer material (silicone, urethane, etc.) that completely encapsulates electronics and comprises the body and housing of the headband, yet allows the headband cover to be flexible so that the headband can form a loop for fitting the head of the user.

Some of the desired features of the headband covering 3 that may be incorporated in the device include that it be durable and sealable to protect the electronics from impact, abrasion, moisture, and weather; that it provide strain relief for connectors and internal electronics; that it allow flexibility in shape, appearance, allows built in labeling; that it allow for easily cleanable surface (good chemical resistance, poor contaminant adhesion); that it be curable at room temperature (or slightly elevated temp.) which is compatible with the electronics; that it be adjustable; that it prevent excessive bending of the headband 1, which may kink or damage flexible circuitry; and that it be light-weight to allow comfortable head mounting on a user.

The preferred design of the headband covering 3 will typically utilize a self-skinning, microcellular, closed cell urethane foam with an integral in-mold coating. The foam and coating are specifically designed to optimize durability and stain resistance while providing a comfortable, lightweight protective covering for the headband. Alternative designs that meet some or all of the desired objectives could also be utilized. Such alternatives might include a design wherein flexible circuit sections could be left exposed for reduction on weight, the band would not be continuous but would instead have segments which match the electronics modules underneath, and that the device be waterproof, allowing for immersion, so that patients can shower, walk outside in rain, etc.

Further, it is preferred that the headband 1 be somewhat raised from head, perhaps by ¼″ to ½″, to allow access to electrode sites as well as allowing for patient comfort and air circulation. The headband 1 can be raised by using “standoffs” (not shown), which could be made from a soft foam or other conformal material attached externally to the body of the headband and/or can be molded directly into body of the headband; if a soft material (such as closed cell foam) is used the standoff could be made in one piece with the body of the device. The standoffs could be mounted onto an external sleeve, sheath or other eternally fastened structure by means of velcro, a button, or other temporary fastening means. The standoffs could then be disposable. The standoffs could also act as cushioning for patient comfort.

Accordingly, the headband covering 3 both protects the internal components and electronics of the device, while providing a flexible structural support, and at the same time helps the device comfortably and snugly fit the user's scalp.

The headband 1 preferably includes a first headband end 5 and a second headband end 7 which can be connected with each other to form the headband 1 into a loop that is sufficiently circular (or oval) to comfortably and securely encircles the head when worn. The ends can also be disconnected to remove the device. These first and second headband ends 5 & 7 each may have a strap that is preferably constructed of high strength material, such as nylon webbing, for example, for strength and durability. Optionally, insert molding the first and second headband ends 5 & 7 into the body of the headband would lend strength and support to the entire device. Or the first and second headband ends could be included in the external sleeve, for example.

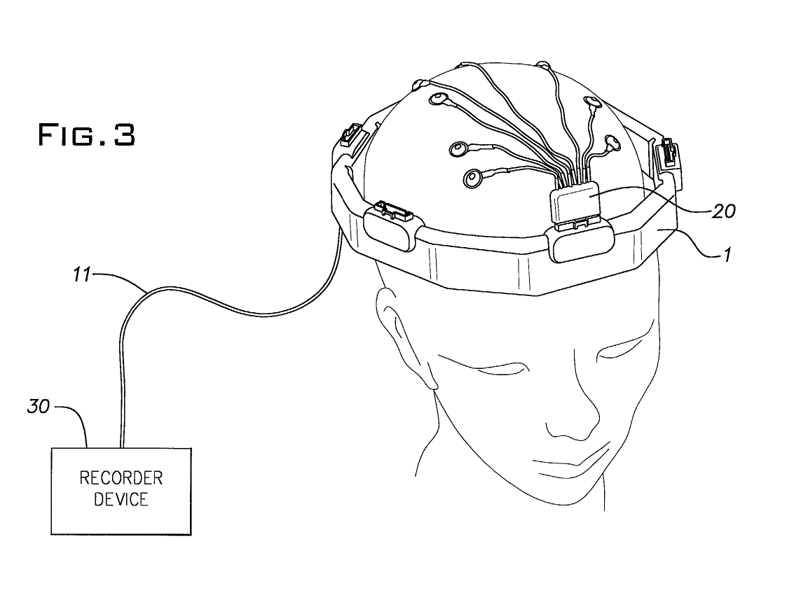

The total lengths of the first headband end 5 and the second headband end 7 are made adjustable, and can be secured together using a means for fastening, such as Velcro or another mechanical fastener such as buttons, snaps, or buckles, for example. Other alternative means of connection are also possible. FIGS. 1 & 2 show one possible implementation of an adjustable fastening means 8, 8′ which provides both fastening and adjusting capability, although other embodiments wherein those functions are provided by separate components is also acceptable. For example, an adjustment mechanism for adjusting the length can be placed on an external sleeve, as part of the strap, or as part of one or both fasteners, for example, as shown in FIGS. 1 & 2. FIG. 3 shows what the medical device might look like when worn by a user when properly adjusted and fastened.

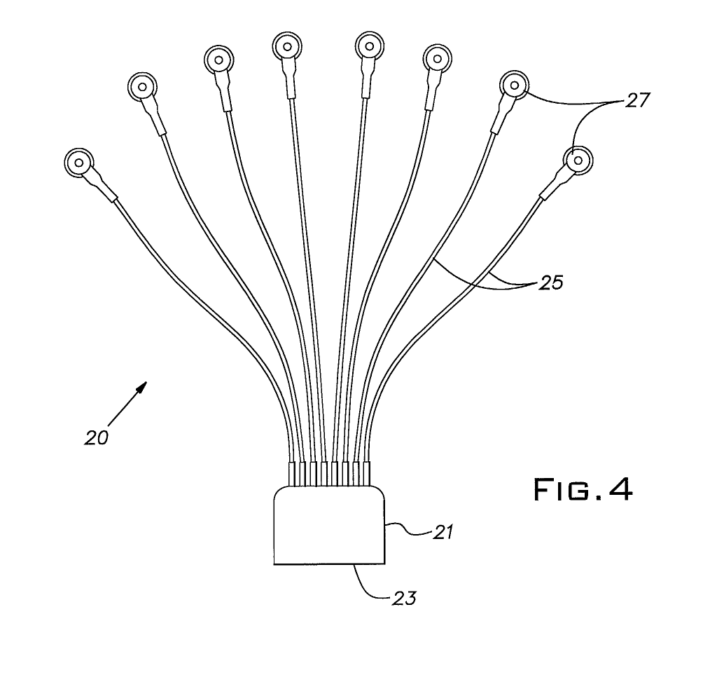

The headband 1 also includes headband connectors 9 for connecting to the electrode assemblies (see FIG. 4, for example). The number of headband connectors 9 depends on the intended application of the device, because each headband connector 9 is designed to support a plurality of electrodes 27. The electrodes can be EEG electrodes, EKG electrodes, or EOG/EMG (electro-oculogram, eye movements) electrodes, for example. In a preferred embodiment, 4 headband connectors are used, as discussed later in this section.

The headband 1 also includes a headband cable 11 for connecting to an external receiving device, such as a biosignals recording device (such as the Biosignals Recorder) or a computer or some other recording or monitoring device.

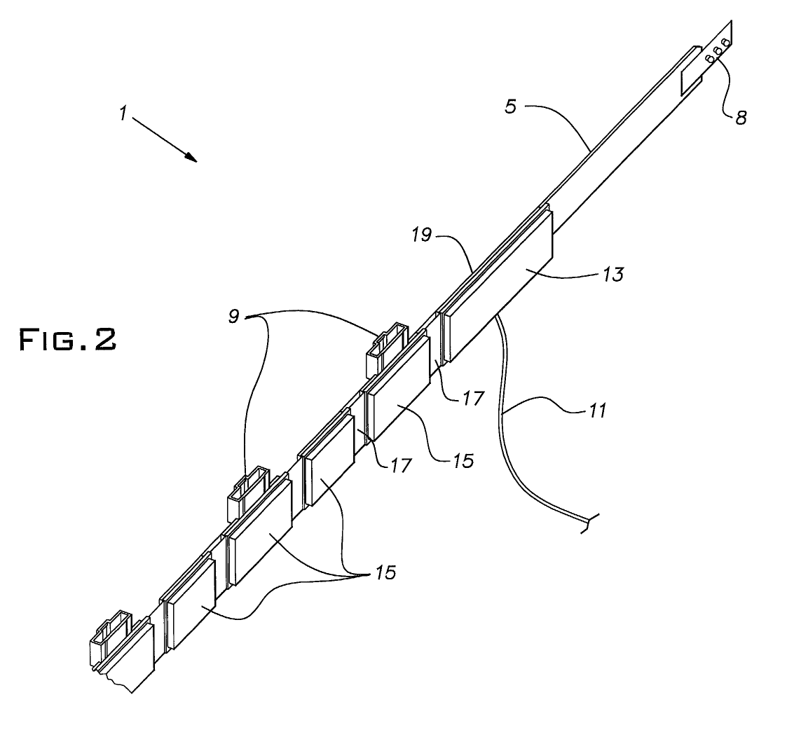

FIG. 2 shows the headband 1 without the external headband covering 3, thus exposing the interior structure of the headband 1. Shown are a plurality of modules including electronic modules 15 which contain signal test/impedance check circuits and signal conditioning circuits mounted on a printed circuit board, as discussed later in this section. Some portion of the electronic modules 15 contain a connector 9 connected thereto, as shown. Also included is at least one processor module 13 containing a microprocessor or controller for overall device control and additional signal conditioning, and, if necessary, a power conditioning and distribution circuit, also described in greater detail later in this section, all mounted on one or more printed circuit boards.

The modules 13, 15 are mounted on a flexible substrate 17 which electrically and physically connects the modules 13, 15 together. The flexible substrate 17 is flexible, and because the modules 13, 15 are typically mounted with gaps between them, the flexible substrate 17 allows the headband to be flexed and/or curved at the gaps so that it can be formed into a somewhat circular band (held together by the connectors of the ends 5, 7) so that the user can wear the device on his or her head. In contrast, the modules 13, 15 are themselves not typically flexible, but in at least one embodiment they are comprised of rigid circuit boards with electronic devices mounted thereon. However, flexible modules could be utilized as an alternative, if they are found to be sufficiently durable to allow the mounting of the appropriate electrical components.

However, in a preferred embodiment, the headband 1 is composed of alternating substantially rigid modules 15 and/or 13 (i.e., they are typically stiff and flexing them might damage them) mounted on the flexible substrate 17 with gaps between them wherein the substrate can be flexed at the gaps, creating an overall rigid-flex design. The advantages of such a design are, for example, that the rigid circuit boards can help protect the delicate electronics devices and avoid open and short circuits, while the flexible substrate allows the device to flex sufficiently so that the device can be adjustable and can mostly conform to the contours of the head, allowing a good compromise between total comfort and durability and reliability.

FIG. 3 shows the headband 1 as worn by an individual user. Also shown in this figure is an electrode assembly 20 with the individual electrodes, such as EEG electrodes, for example, shown as they might be mounted on a user's scalp, for an example use. Typically, more than one electrode assembly 20 would be connected to the headband 1. The electrode assembly 20 can also be disconnected from the headband 1. Each electrode assembly has an electrode connector 23 (shown on FIG. 4) which physically and mechanically connects to the headband connector 9 (shown on FIGS. 1 and 2). The connectors 23 & 9 should allow for the ease of replacing an electrode assembly, if necessary (e.g., if one or more electrodes fail).

Note that the device plugs into an external receiving device such as the recorder unit 30 via the headband cable 11. The recorder unit 30 might be mounted on a belt, the user's chest, or carried in the user's pocket, for example. The headband cable 11 carries the headband data output signal to be recorded or monitored by the external receiving device, and may also carry commands and/or power back to the medical device.

FIG. 4 shows the electrode assembly 20, shown with the electrode connector 23, with multiple electrode leads 25 emanating from the electrode connector 23, and with each lead 25 connecting to one or more individual electrodes 27 (such as EEG electrodes, EKG electrodes, or EOG/EMG electrodes, for example). The electrode assembly is preferably scaleable, allowing many different combinations and numbers of electrodes and electrode assemblies to be utilized. The most useful configuration will likely utilize 4 headband connectors 9 on the headband 1, each connector 9 mounted on an electronic module, each connector 9 connecting to the electrode connector 23 of an electrode assembly 20, each electrode assembly 20 typically with 6 or more electrode leads 25 and corresponding electrodes 27, although different numbers of electrodes are easily supported, such as 4, 8 (as shown in FIG. 4) or 11, for example.

The electrodes 27 are typically attached to the body of the user, for example, to the scalp in the case of an EEG application (see FIG. 3, for an example) or to the chest for EKG/ECG applications, or to the eye regions for EOG/EMG applications. It is expected that one or more electrodes will be used as a ground terminal (that may be attached to the scalp, or alternatively to another part of the body, such as an ear or the chest, for example) for connection to the ground plane of the medical device. Further, one electrode is expected to be used as a reference electrode, explained in more detail later in this section.

Surface electrodes with silver/silver chloride electrodes or gold plated metal electrodes that adhere to the patient's skin, are the preferred electrode design. The off-the-shelf versions of these electrodes will likely suffice, although the leads may need to be shortened.

The total number of connectors used can be varied based on the desired number of leads and the intended use of the device. Further, electrodes can be color coded or otherwise marked to aid in placement and recognition, and further benefit might be obtained by making the headband graduated or otherwise marked to aid in electrode placement. The connectors can be easily removed and replaced as necessary, especially if an electrode lead or electrode goes bad, allowing partial replacements of electrodes and easy repair. The use of electrode assemblies helps prevent accidental electrode disconnection and helps prevent cable snags, in contrast to other devices that use only individual electrodes. The body of the device supports the connectors and provides strain relief, and the single small data cable 11 from the headband 1 is used to connect to the recording device or other receiver.

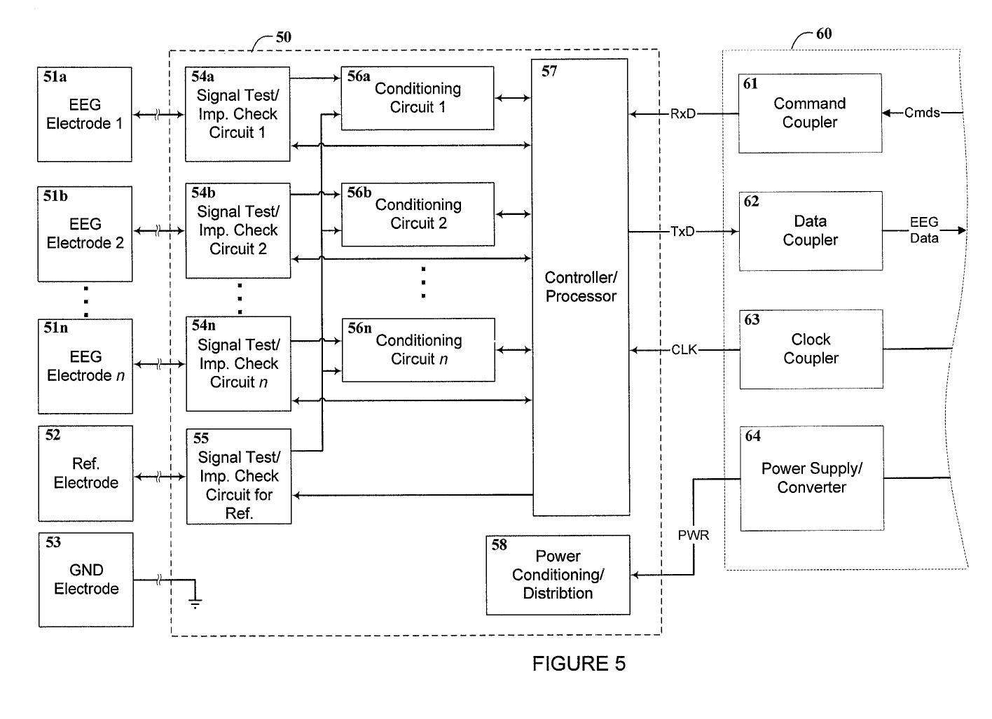

FIG. 5 shows a block diagram of a preferred embodiment of the electronic components of the headband 1. A headband circuit 50 is comprised of additional circuits that include the data conditioning and processing portions of the medical device, while the external receiving device 60 shows a portion of the likely processing and power components of the external receiving device (such as the Biosignals Recorder, for example), if connected to the headband 1. The headband circuit 50 transmits (TxD) a headband data output signal to a data coupler 62 of the external receiving device 60. The headband data output signal is preferably a serial data signal. A command coupler 61 of the external receiving device may transmit commands to the headband circuit (RxD). A power supply/converter device 64 may also be a part of the external receiving device 60 to provide power to the headband circuit.

The electrodes 27 of the device are represented by the EEG electrode blocks 51a, 51b, to 51n for “n” total EEG electrodes which represent “n” total numbers of channels of the device. However, the device also supports EKG electrodes and/or EOG/EMG electrodes as well. [0055] Typically, an EEG application might use anywhere from 4 to 128 or more channels, depending on the application and purpose of the device and the medical condition being monitored.

In a typical embodiment, there might be 32 channels, along with an additional reference electrode and ground electrode (The reference electrode is represented in FIG. 5 by block 52 and the ground electrode by block 53), leading to 34 total electrodes. All 32 channels might be used for EEG monitoring, or alternatively, some channels could be used for EEG monitoring and others for EKG monitoring, or EOG/EMG monitoring, or some combination of all three. At least one preferred embodiment uses 30 EEG channels and 2 EKG channels for monitoring both EEG and EKG simultaneously. However, many other combinations are possible and within the scope of the device.

The electrodes could be organized, for example, into two groups of 6 electrodes and two groups of 11 electrodes, each group with its own electrode assembly. The headband would then have at least 4 corresponding headband connectors which would electrically and mechanically connect to each of the electrode connectors. Thus, each electrode connector mates with a corresponding headband connector, one of which would have a “male” connector and the other of which would have a corresponding “female” connector to provide both the mechanical and electrical connection between the headband 1 and the electrode assembly 20. A mechanical connection that allows for ease of replacement of an electrode connector, should an electrode go bad, is preferable.

The EEG electrodes generate an electrode signal based on activities and processes occurring in the human body (such as brain waves for EEG and heartbeats for EKG/ECG, for example). In the preferred embodiment, an analog electrode signal is generated. Thus, each electrode generates a corresponding electrode signal that travels along the corresponding electrode lead to a corresponding electrode connector for input into the headband via the headband connector.

The headband circuit 50 includes the capability of checking the impedance of each electrode and calibrating amplification of the electrode signal. FIG. 5 shows a preferred embodiment wherein each electrode 51a, 51b . . . 51n has its output connected to a corresponding signal test and impedance test circuit 54a, 54b . . . 54n, into which the electrode signal from the corresponding electrode is input. The signal test and impedance test circuits 54 are used to test and check the electrical characteristics of each electrode and the amplification gain of the amplifier(s) of the conditioning circuits. The electrode signal is also input into a corresponding conditioning circuit 56a, 56b . . . 56n.

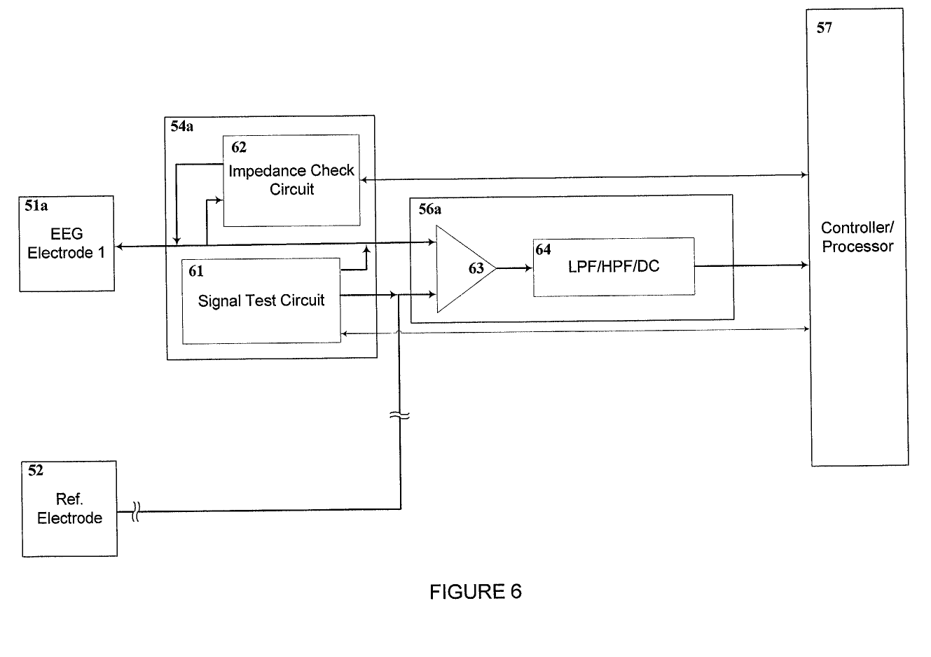

In a preferred embodiment, the signal test and impedance test circuit 54 design includes a signal test circuit 61 and an electrode impedance check circuit 62, as shown in FIG. 6. In this preferred embodiment, each signal test circuit sends a signal test test signal to the corresponding conditioning circuit to determine an amplification gain. Preferably, the signal test test circuit(s) utilize a group of single pole single throw (SPST) analog switches that (once activated by a controller/processor 57, described later in this section) will connect the signal input of an input amplifier of each conditioning circuit to ground through a resistor (such as a 1 kohm resistor, for example), while at the same time a known signal test pulse originating from the controller/processor 57 will be directed into the reference input of each amplifier. The electrodes are disconnected from the patient at signal test time. In a preferred embodiment, the signal test circuit generates a pulse or a sine wave (at, for example, 5 Hz) with a fixed voltage signal (at, for example, 0.5 mV). The fixed voltage signal feeds into each channel and the output from each channel is measured. The expected amplification gain of the circuit is known, at 2000, for example, so an output voltage of 1000 mV is expected if the input voltage is 0.5 mV. Anything less than or higher than the expected output will mean the gain is not at the proper level. Thus, the measured output indicates whether an amplification gain adjustment for any channel or channels is desirable, and the amplification gain can be adjusted as needed, manually, or perhaps automatically. Further, the gain error could instead be compensated for in any calculations or data transformations of the signals.

Alternatively, a single signal test circuit might be utilized to check the amplification gain of the conditioning circuit of each channel by switching the circuit to connect to each conditioning circuit individually, for example. This could beneficially reduce the circuit complexity in some circumstances.

Also preferably included is a safety switch (such as an SPST analog switch, for example, not shown) to electrically disconnect the reference signal from the headband, thus opening the return path and thereby isolating the user from the signal test pulse

The electrode impedance check circuit 62 (E-Check) measures the EEG electrode impedance of each electrode to ensure that it is properly placed on the patient. The impedance check circuit does this by sending an impedance test signal to the EEG electrode. A low voltage sine wave is applied to the input of each conditioning circuit (and thus the electrode output) to check the electrode impedance. The impedance check circuit generates a very small current (less than 100 micro amps at 5 Hz, for example) to be input into each electrode, and the circuit simultaneously measures the resultant voltage at the electrodes. The resultant voltage at the electrode lead is indicative of electrode impedance. If it is too high (greater than 3 V in the example) then the electrode is not properly connected. If it is low (less than 3 V in the example) the electrode is properly connected.

Alternatively, a single impedance check circuit might be utilized to check the impedance of each electrode by switching the impedance check circuit to connect to each electrode individually, for example. This could beneficially reduce the circuit complexity in some circumstances.

The electrode signal is processed and conditioned by the headband circuit 50. Note that in the preferred embodiments the electrode signals are primarily EEG signals, but there may also be some combination of EKG and/or EOG/EMG electrode signals supported as well. Thus, the headband circuit is not limited to processing only EEG signals, but can process all electrode signals. Processing and conditioning the electrode signals preferably includes one or more of filtering, amplification, DC offset clamping, and A/D conversion of each electrode signal, and multiplexing the various channels together. FIG. 5 shows the output of each electrode 27 is input into a corresponding conditioning circuit 56a, 56b . . . 56n, respectively. The conditioning circuits 56 condition the electrode signals, providing filtering and amplification of the signals, as shown in FIG. 6, sufficient for inputting conditioned signals, the number corresponding to the number of channels of EEG electrodes, into a processor module 57 to allow for processing of the conditioned signal and thus outputting a processed signal. Processing might include analog-to-digital (A/D) conversion, further filtering, and/or multiplexing, for example. The conditioning circuits each utilize a common reference signal, described below.

The reference electrode 52 preferably has its own signal test and impedance test circuit 55. The output of the reference electrode is a reference signal input into each conditioning circuit 56a, 56b . . . 56n, to be used as a common reference signal. Thus, the reference electrode 52 does not typically use its own conditioning circuit. The reference electrode may be connected to the scalp, other portions of the head (such as an earlobe, for example), or some other part of the body, as desired.

The ground electrode 53, however, is connected to the electrical ground plane of the device, and does not typically require any signal test or impedance check circuit, nor any signal conditioning. The ground electrode may be connected to the scalp, other portions of the head (such as an earlobe, for example), or some other part of the body, as desired.

The signal test and impedance test circuits 54a, 54b . . . 54n and 55 along with the conditioning circuits 56a, 56b . . . 56n, are distributed among various of the electronic modules. The actual distribution is not critical to the design, allowing for various configurations to be used providing flexibility in the design and layout. In at least one embodiment, the signal test and impedance test circuits are grouped such that 4 channels are handled by each electronic module. However, different combinations and arrangements would be acceptable as well.

As FIG. 6 shows, the signal conditioning circuits 56 preferably have one or more amplifiers 63 that amplify the analog (and/or digital signals, which might be accommodated) received from the transducers. Each conditioning circuit preferably consists of at least one instrumentation pre-amplifier, and a filtering circuit that provides low pass filtering, high-pass filtering, and DC offset clamping. However, different filtering operations may be implemented depending on the purpose of the device. The specific filtering and amplification implementations utilize circuits and components known in the art, and thus not detailed here. The conditioning circuit typically outputs an analog conditioned signal derived from the analog electrode signal input.

The amplified signal can, in addition, be further filtered, multiplexed, and/or further amplified again by additional circuits of the conditioning circuits 56, or, alternatively, some or all of these functions can be performed by the controller/processor 57.

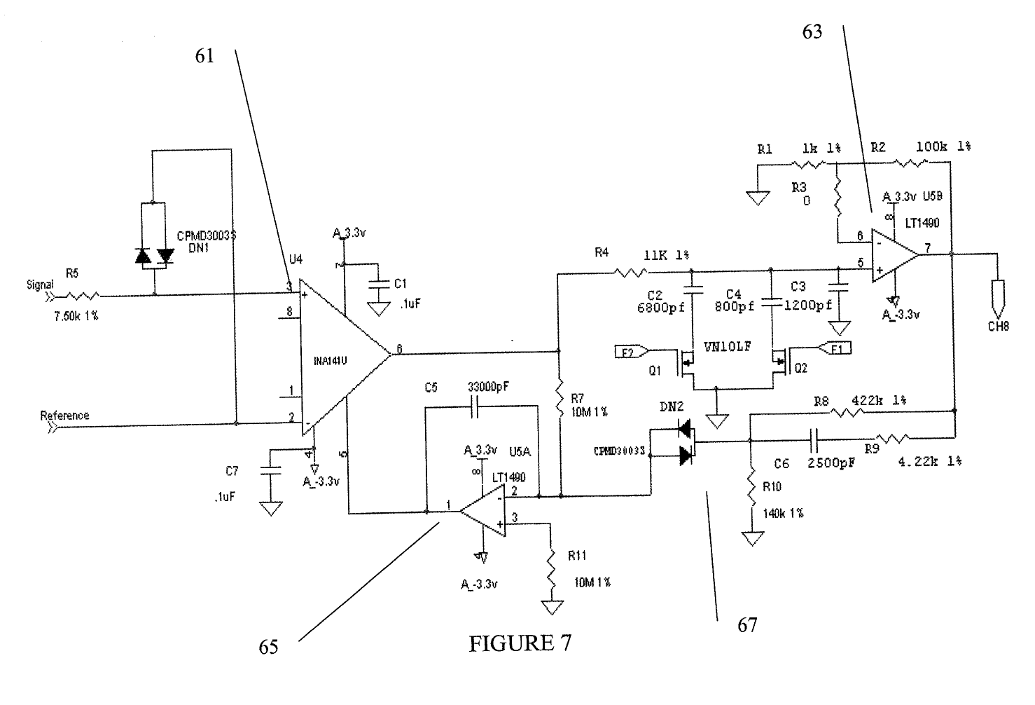

FIG. 7 shows in more detail the circuitry for amplifying, low pass filtering, and DC offset clamping the electrode signal. The circuit has an amplifying portion 61, a low pass filtering portion 63, a high-pass filtering portion 65, and a DC offset clamping portion 67. The DC Offset clamping circuit gives the conditioning circuit the capability to limit DC shifts in the EEG signal by implementing a DC clamping circuit on each channel. This alleviates DC shift problems that often arise during EEG procedures when patients move around during monitoring, causing the cables to move.

The resulting signal will preferably then undergo an analog-to-digital conversion in the headband circuit, typically using a high-resolution A/D process, if the conditioned signal is in analog form (as is anticipated to be the typical case). The A/D conversion will transform the individual or multiplexed analog signals into a stream of digital data bits for storage. The number of bits representing each multiplexed signal (or channel) depends on the A/D unit and/or the desired fidelity, and can be 8, 10, 12, or 16 or some other useful number of bits. Increasing the number of data bits can increase the resolution of the data being processed. In the preferred embodiment, the controller/processor 57 will perform the A/D function.

FIGS. 5 & 6 further show the controller/processor 57. The controller/processor 57 will likely utilize a general purpose microprocessor or microcontroller of sufficient speed to perform various functions. The functions are typically implemented by programming the controller/processor with software (or firmware) or activating functions embedded therein in off-the-shelf firmware or hardware. The functions likely to be performed by the controller/processor include analog-to-digital transformation of the EEG conditioned signals to obtain EEG digital signals. The controller/processor 57 (or additional circuits) could further condition the conditioned signals (such as additional amplification and/or filtering, for example) before (or even after) performing the A/D operation, if desired. The controller/processor 57 (or other electrical circuits) may also, if desired or needed, perform additional signal processing of the digital signals (such as data compression, for example), and the controller/processor or, alternatively some other circuit, will then multiplex the processed data signal of each channel for ease of transmission of the headband data output signal to the external receiving device. Preferably, the headband data output signal will be a serial data stream.

Alternatively, the device can also be implemented to utilize digital electrode signals, wherein all filtering and amplification would be digitally implemented, and no A/D conversion would be necessary.

The resulting EEG data signal is then output to the external receiving device, such as the shown recording device 30, via the headband cable 11 (see FIG. 3, for example, showing this arrangement). The headband cable 11 is likely to be a four or more conductor cable, with a serial data connection to the recording device to carry the multiplexed output of the controller/processor, and/or commands back to the controller/processor, and perhaps also having a power connection to provide power to the device.

The output of the device is preferably serial, being output from the controller/processor unit 57 and fed into an external device with the capability to read the serial data. Preferably, the data will be input to a data coupler device 62, as shown in FIG. 5, to isolate the external device from the medical device. The external receiving device could be utilized to provide all necessary commands and control signals to the medical device, such as via the command coupler 61 shown in FIG. 5, to ensure that the data is properly acquired and the device is working properly. These command and control signals can be utilized to start and stop acquisition, control the number of channels to be acquired, check electrode impedance (via the electrode impedance check circuitry), and check for circuit operation (via the signal test circuitry). The external device can be a PC, or a recorder or other data storage device, such as the Biosignals Recorder.

Further, the Head Mounted Medical Device may utilize a wireless data transmission capability to broadcast the headband data output signal directly to a remote receiver of the external device, which could be a remote computer or a relay device. The remote receiver might also be able to broadcast information back to Head Mounted Medical Device. Bluetooth wireless technology could be utilized, which uses small broadcasting chips that can be embedded into the medical receiver to broadcast real-time or recorded data to a receiving device, which can then transmit the data to a remote location. Because Bluetooth is a two-way communication technology, information could also be transmitted from a remote location to Head Mounted Medical Device to provide the ability of a medical worker to interact with the device. Cellular technology is another means for broadcasting information to and from the recording unit. If such technology is utilized, security measures must be implemented, such as password control and encryption keys, for example, to protect the patient's medical data and to prevent unauthorized access to the recording device.

The Head Mounted Medical Device may or may not contain any batteries or any other power supplies. If no batteries are included the device will be powered from the external receiving device (such as the Biosignals Recorder or another external device) via the headband cable 11. FIG. 5 shows an embodiment wherein power is provided by the external recording device by a power supply or converter 64, for input into a power conditioning and distribution circuit 58 of the headband. If wireless technology is implemented, the Head Mounted Medical Device should be self powered via a battery.

The controller/processor 57, along with the power conditioning and distribution circuit 58 and any necessary electronics to support these components, are preferably to be mounted on a printed circuit board and integrated into the processor module 13 (see FIG. 2). The processor module 13 may be physically implemented using more than one sub-module. In a preferred embodiment, three sub-modules are used, containing the controller/processor, power regulation and conditioning circuitry, and any other support circuitry. The number of sub-modules is chosen based on physical parameters and consideration, such as allowing enough flexibility in the headband to allow it to comfortably conform to the user's head. The headband cable 11 is preferably connected to the processor module 13, or thereabouts, for convenient reasons and noise minimization.

Although the design of the device modules is flexible, in a preferred embodiment, the device is split into 11 total modules, eight of the 11 modules being electronic modules containing, for example, 4 channels each, with a signal test and impedance test circuit and a conditioning circuit for each channel, for a total of 32 channels, while three of the modules are sub-modules encompassing the processor module. With the addition of reference and ground electrodes to one or two of the modules, that leads to 34 total electrodes. Because in the preferred embodiment there are 4 headband connectors supporting 4 electrode assemblies, 4 of the modules must have headband connectors mounted thereon. These are typically distributed across the headband, as shown in FIG. 4, although it may be desirable to have none of them located on the back of the unit, especially if the unit is to be worn while the user is sleeping, for comfort reasons.

Of course, the modules and the components thereon can be alternatively re-arranged in any number of configurations while maintaining consistency with this disclosure, as the distribution of components among the modules, and the distribution of modules across the headband, is sometimes more a matter of convenience and design choice than it is functionally determined, although considerations such as user comfort, the flexibility of the resulting headband to form a band, noise and interference issues, along with limitations on electronics miniaturization, may all be important considerations in determining this arrangement. The use of 11 total modules has shown to provide a good compromise among all these criteria.

Each electronic module preferably utilizes a very close and well-organized placement of components to create a highly efficient, low noise layout. The preferred layout utilizes a total of 8 layers: Top, +Vdc, Input & E-Check, Ground, Reference, −Vdc, Output, and bottom layers, preferably arranged as described in the table below:

| | | |
|---|---|---|
| 1 | Components | Route Layer |
| 2 | +Vdc | Plane Layer |
| 3 | Input & E-Check signals | Route Layer |
| 4 | Ground | Plane Layer |
| 5 | Reference Layer | Plane Layer |
| 6 | −Vdc | Plane Layer |
| 7 | Output signals | Route Layer |
| 8 | Components | Route Layer |

The power and ground planes are used to eliminate ground loops, eliminate EMI as well as minimize routing lengths. The Reference plane is a layer dedicated to the Reference inputs to each channel. Because the reference signal is common to each channel (and input to each channels conditioning circuit), this plane makes routing more efficient, as every channel drops a short trace instead of using a daisy-chain routing method.

The Input and E-Check layer is placed between two solid planes, +Vdc and ground, for better noise immunity. The Reference layer is also placed between two solid planes, −Vdc and ground, for noise immunity. The Output signals are preferably at a higher voltage than the input signals by a magnitude up to 1000, and are thus less susceptible to noise; therefore it is not critical that it be placed between two solid planes. The Top and Bottom planes contain the components and routing between components for each channel.

The Component placement and Layout method presents the possibility of a scaleable design, dependent on the amount of components loaded on the board the headband can have either 4, 8, 16, 24 or the full 32 amplifier channels or even more, depending on the desired application.

The headband data output signal transmitted by the controller/processor via the headband cable 11 will preferably be in serial form and can transmit at high data rates (possibly 1 Mbps or more) allowing the use of the Head mounted EEG device in applications such as ambulatory EEG monitoring where low sampling rates are sufficient and routine EEG applications where high sampling rates are needed. The Head Mounted Medical Device can also be used to provide the EEG signals for long term monitoring (LTM) or sleep studies where high signal fidelity and less obtrusive devices are needed. Each channel can be sampled at around 500 Hz to 2 kHz or more, as needed, providing high-fidelity data collection.

The device will maintain ease of hookup. The Head Mounted Medical Device will incorporate multiple lead connectors which plug into the headband. These connectors will allow the use of the same electrode types used conventionally but using shorter lead wires, thereby providing benefits in noise reduction and ease of use. Technicians could place the electrodes on the head one electrode at a time without having to worry about affecting the location of other electrodes or the headset. The Head Mounted Medical Device may be engraved with measurement marks on the periphery to aid the technician in electrode placements.

The Head Mounted Medical Device can interface with various medical transducers and/or recorders, depending on the application or medical condition being monitored. Such transducers could be commercial, off-the-shelf devices. For instance, an EEG transducer (such as the electrodes 27 shown in FIG. 4) and/or an EKG transducer may consist of a set of electrodes, which are typically surface electrodes with silver/silver chloride electrodes or gold plated metal electrodes, that adhere to the patient's skin. The EKG transducer might use 5, 7, or 10 electrodes, while the EEG transducer might use up 128 electrodes in many different configurations (ranging from 2, 8, 16, 32, 64, & 128 or more electrodes). Some specific EKG/EEG recordings conducted for research purposes can even use up to 256 electrodes or more. Each electrode can be connected to the signal conditioning circuit of the invention via the headband/electrode assembly connectors. Additional medical transducers can also be accommodated, because the device can be adapted to function with many different medical transducers.

A common generic interface to an external recorder unit or other monitoring device could be developed that would then provide an interface to the various transducers. Such an interface could be made external to the recorder device if that would be advantageous in a particular situation. If an external interface is used, that interface could be made wireless, thus eliminating any tethering or restrictions on where to place the device in relation to the patient.

The device could also be capable of storing a medical event as experienced by the user. An “event” button placed on the outside housing of the device will allow the user to indicate a potential episode (cardiac arrhythmia or epileptic seizure, for example). After the button is pressed, the device will be able to embed a real time marker in the EEG data to be recorded or identified by the external device.

The device might also store data via solid state miniature hard drives, optical disks, or any other storage media making it possible for the device to be independent from the external receiving device. Such uses could be beneficial in ambulatory (e.g., home or office) conditions or under hospital conditions. Such recording could be short term (such as when the device is temporarily unhooked from the monitor in the doctor's office) or long-term (when the patients take the device home, to work, or during other daily activities, where no monitors may be available.)

A number of medical devices utilizing this new head mounted medical device can be developed according to the invention as herein disclosed. The invention has been described using specific examples; however, it will be understood by those skilled in the art that various alternatives may be used and equivalents may be substituted for elements described herein, without deviating from the scope of the invention. Modifications may be necessary to adapt the invention to a particular situation or to particular materials without departing from the scope of the invention. It is intended that the invention not be limited to the particular implementation described herein, but that the claims be given their broadest interpretation to cover all embodiments, literal or equivalent, covered thereby.

What is claimed is:

1. A head mounted medical device comprising:
an electrode for outputting an electrode signal;
a circuit for inputting and conditioning said electrode signal into a data signal for output by said medical device into an external receiving device;
at least one module including some portion of said circuit; and
a flexible substrate for mounting said at least one module thereon, wherein said flexible substrate can be formed into a loop for mounting on the head of a user.

2. The device of claim 1, further comprising at least one additional module, wherein said circuit is distributed among said modules and further wherein said flexible substrate electrically connects at least two of said modules together.

3. The device of claim 2, wherein said modules are substantially rigid, and further wherein said modules are mounted on said flexible substrate with gaps between said modules such that said flexible substrate can be formed into a loop by flexing said flexible substrate at the gaps.

4. The device of claim 2, further comprising:
at least 3 additional electrodes each for outputting a corresponding electrode signal; and
a plurality of electrode assemblies, each electrode assembly including at least two electrodes and an electrode connector, wherein said electrode connector electrically and mechanically connects to a corresponding headband connector mounted on a module;
wherein said circuit is further for inputting, conditioning, and multiplexing said electrode signals into said data signal.

5. The device of claim 4, wherein said modules are substantially rigid, and further wherein said modules are mounted on said flexible substrate with gaps between said modules such that said flexible substrate can be formed into a loop by flexing said flexible substrate at the gaps.

6. The device of claim 1, further comprising:
at least 3 additional electrodes each for outputting a corresponding electrode signal; and
a plurality of electrode assemblies, each electrode assembly including at least two electrodes and an electrode connector, wherein the electrode connector electrically and mechanically connects to a corresponding headband connector mounted on a module;
wherein said circuit is further for inputting and conditioning said corresponding electrode signals into said data signal.

7. The device of claim 1, said circuit including:
a controller/processor;
a conditioning circuit for inputting and conditioning and amplifying said electrode signal and also for outputting a conditioned electrode signal into an s input of said controller/processor, wherein said controller/processor converts said conditioned electrode signal into said data signal.

8. The device of claim 7, said circuit further including an impedance check circuit for outputting an impedance test signal to said electrode to determine an electrode impedance of said electrode, wherein said controller/processor monitors said electrode impedance.

9. The device of claim 8, said circuit further including a signal test circuit for outputting a signal test test signal into said conditioning circuit to determine an amplification gain of said conditioning circuit.

10. The device of claim 1, said circuit including an impedance check circuit for outputting an impedance test signal to said electrode to determine an electrode impedance of said electrode.

11. The device of claim 1, said electrodes including a plurality of EEG electrodes, each outputting an EEG electrode signal; and a plurality of EKG electrodes, each outputting an EEG electrode signal.

12. The device of claim 1, wherein said circuit includes a DC offset clamping circuit for clamping a DC offset in said electrode signal.

13. A head mounted medical device comprising:
a plurality of electrodes, each electrode for outputting an electrode signal;
a headband circuit for inputting said electrode signals; wherein said headband circuit amplifies, filters, and multiplexes said electrode signals, further generating a headband data output signal therefrom for output by said device into an external receiving device;
an electrode assembly including two or more electrodes;
a plurality of modules, each module including a portion of said headband circuit;
wherein said electrode assembly can be mechanically and electrically removably connected to at least one of said plurality of modules; and
a flexible substrate for mounting said plurality of modules thereon, said flexible substrate electrically connecting at least some of said plurality of modules together, wherein said flexible substrate can flex, allowing said flexible substrate to be formed into a loop for mounting on the head of a user.

14. The device of claim 13, wherein each electrode signal is an analog electrode signal, and further wherein said headband circuit converts each analog electrode signal into a digital format all being multiplexed together into said EEG data signal output by said device, said EEG data signal being a serial data signal.

15. The device of claim 13, further comprising:
one or more additional electrode assemblies, each electrode assembly including at least two electrodes and an electrode connector, wherein each electrode connector electrically and mechanically connects to a corresponding headband connector mounted on one of said modules.

16. The device of claim 13, said headband circuit including:
a controller/processor;
a plurality of conditioning circuits, each conditioning circuit for inputting and conditioning and amplifying a corresponding electrode signal and also for outputting a conditioned electrode signal into an input of said controller/processor, wherein said controller/processor converts said conditioned electrode signals into said EEG data signal.

17. The device of claim 16, said headband circuit further including at least one impedance check circuit for outputting an impedance test signal to each electrode to determine an electrode impedance of said electrode, wherein said controller/processor monitors said electrode impedance of each electrode.

18. The device of claim 17, said headband circuit further including at least one signal test circuit for outputting a signal test test signal into each conditioning circuit to determine an amplification gain of each conditioning circuit.

19. The device of claim 13, said headband circuit including at least one impedance check circuit for outputting an impedance test signal to each electrode to determine an electrode impedance of said electrode.

20. The device of claim 13, wherein said modules are substantially rigid, and further wherein said modules are mounted on said flexible substrate with gaps between said modules such that said flexible substrate can be formed into a loop by flexing said flexible substrate at the gaps.

21. The device of claim 13, said electrodes including a plurality of EEG electrodes and a plurality of EKG electrodes, wherein said electrode signal is an EKG electrode signal when outputted from any EKG electrode and further wherein said electrode signal is an EEG electrode signal when outputted from any EEG electrode.

22. The device of claim 13, wherein said headband circuit is also for clamping a DC offset in said electrode signal.

23. A head mounted medical device comprising:
a controller/processor;
a plurality of channels, each channel including:
an electrode for outputting an electrode signal; and
a conditioning circuit for inputting and conditioning said electrode signal; said conditioning circuit also for outputting a conditioned electrode signal into an input of said controller/processor, wherein said controller/processor processes said conditioned electrode signal into a processed signal;
wherein said controller/processor multiplexes each processed signal together into a headband data output signal for output by said device into an external receiving device;
at least one electrode assembly including two or more electrodes and an electrode connector;
a plurality of modules, each module including one or more of said controller/processor and said conditioning circuits;
wherein said electrode connector can be mechanically and electrically removably connected to a corresponding headband connector mounted on at least one of said plurality of modules; and
a flexible substrate for mounting said plurality of modules thereon, said flexible substrate electrically connecting at least some of said plurality of modules together, wherein each of said plurality of modules is mounted some distance from another of said modules creating gaps between said modules, said gaps allowing said flexible substrate to flex at the gaps, allowing said flexible substrate to be formed into a loop for mounting on the head of a user.

24. The device of claim 23, further comprising at least one impedance check circuit for outputting an impedance test signal to each electrode to determine an electrode impedance of each electrode, wherein said controller/processor monitors the electrode impedance of each electrode.

25. The device of claim 23, further comprising at least one signal test circuit for outputting a signal test test signal into each conditioning circuit to determine an amplification gain of each conditioning circuit.

26. The device of claim 23, said electrodes having a plurality of EKG electrodes and a plurality of EEG electrodes, wherein said electrode signal is an EKG electrode signal when outputted from the EKG electrode and further wherein said electrode signal is an EEG electrode signal when outputted from the EEG electrode.

27. The device of claim 23, wherein said conditioning circuit is further for clamping a DC offset in said electrode signal.

28. A head mounted medical device comprising:
a reference electrode for mounting on the scalp, head, or body of a user and also for outputting a reference signal;
a ground electrode for mounting on the scalp, head, or body of said user, said ground electrode connected to a ground plane of said medical device;
a controller/processor;
a power conditioning circuit for conditioning and distributing power to said device;
a plurality of channels, each channel including:
an EEG electrode for mounting on the scalp of the user and also for outputting an analog electrode signal;
an impedance check circuit for outputting an impedance test signal to said EEG electrode to determine an EEG electrode impedance of said EEG electrode, wherein said controller/processor monitors said EEG electrode impedance;
a conditioning circuit for inputting and conditioning said analog electrode signal, said conditioning including amplifying, filtering, and clamping a DC offset; said reference signal also being input to said conditioning circuit; said conditioning circuit also for outputting a conditioned electrode signal into an input of said controller/processor, wherein said controller/processor converts said conditioned electrode signal into a digital electrode signal; and
a signal test circuit for outputting a signal test test signal into said conditioning circuit to determine an amplification gain of said conditioning circuit;
wherein said controller/processor multiplexes each digital electrode signal together into a headband data output signal for output by said medical device into an external receiving device, and further wherein said controller/processor is capable of receiving commands from said external receiving device;
a plurality of electrode assemblies, each electrode assembly including two or more EEG electrodes and an electrode connector;
a plurality of modules, each module including at least one printed circuit board; each module also including, mounted on said at least one printed circuit board, one or more of the group consisting of said impedance check circuits, said signal test circuits, said conditioning circuits, said power conditioning circuit, and said controller/processor;
wherein said electrode connector can be mechanically and electrically removably connected to a corresponding headband connector mounted on at least one of said plurality of modules;
a flexible substrate for mounting said plurality of modules thereon, said flexible substrate having a first end and a second end, said flexible substrate electrically connecting at least some of said plurality of modules together, wherein each of said plurality of modules is mounted some distance from another of said modules, creating gaps between said modules, said gaps allowing said flexible substrate to flex at said gaps, allowing said flexible substrate to be formed into a loop for mounting on the head of the user, wherein said first end and said second end are removably connected together using an adjustable fastening means for securing said first end to said second end, said adjustable fastening means allowing said flexible substrate to be adjusted to fit the head of the user; and
a headband cover for covering and protecting said modules and said flexible substrate, said headband cover being flexible.

29. The device of claim 28, wherein said power conditioning circuit receives power from said external receiving device.

30. The device of claim 28, said plurality of channels further including a plurality of EKG electrodes, wherein said electrode signal is an EKG signal when outputted from the EKG electrode and further wherein said electrode signal is an EEG signal when outputted from the EEG electrode.

31. The device of claim 30, wherein said headband device comprises 32 total channels, two of which are EKG channels and 30 of which are EEG channels, and further wherein said plurality of modules includes:
8 electronic modules for mounting 32 conditioning circuits, 4 of said 8 electronic modules having a headband connector for connecting to corresponding electrode connectors included in said electrode assemblies
34 impedance check circuits;
32 signal test circuits; and
3 processor modules for mounting said controller/processor and said power conditioning circuit, with an output cable connected to one of said 3 modules, said output cable having less than 8 conductors, said output cable for connecting to said external receiver device and for carrying said multiplexed EEG serial signal and said commands;
wherein said EEG device further comprises 4 electrode assemblies each for connecting to a different one of said headband connectors on each of said 4 of said 8 electronic modules, each electrode assembly with 6 or more of said reference electrode, said ground electrode, and said EEG electrodes.

32. The device of claim 28, further comprising a reference impedance check circuit for outputting an impedance test signal to said reference electrode to determine a reference electrode impedance of said reference electrode, wherein said controller/processor monitors said reference electrode impedance;
wherein said medical device comprises 32 channels, and further wherein said plurality of modules includes:
8 electronic modules for mounting 32 conditioning circuits, 4 of said 8 electronic modules having a headband connector for connecting to corresponding electrode connectors included in said electrode assemblies
34 impedance check circuits;
32 signal test circuits; and
3 processor modules for mounting said controller/processor and said power conditioning circuit, with an output cable connected to one of said 3 modules, said output cable having less than 8 conductors, said output cable for connecting to said external receiver device and for carrying said multiplexed EEG serial signal and said commands;
wherein said medical device further comprises 4 electrode assemblies each for connecting to a different one of said headband connectors on each of said 4 of said 8 electronic modules, each electrode assembly with 6 or more of said reference electrode, said ground electrode, and said EEG electrodes.

33. The device of claim 28, wherein each channel is sampled at a rate of at least 500 Hz.

34. The device of claim 28, wherein each channel is sampled at a rate of at least 2 kHz.
